(19) United States
(12) Patent Application Publication
(10) Pub. No.: US 2024/0090905 A1
(43) Pub. Date: Mar. 21, 2024

(54) Elastic Limb Tourniquet

(71) Applicant: Tygran Amalyan, Kyiv (UA)

(72) Inventor: Tygran Amalyan, Kyiv (UA)

(21) Appl. No.: 18/131,243

(22) Filed: Apr. 5, 2023

(30) Foreign Application Priority Data
Sep. 19, 2022 (UA) U 2022 03433

(51) Int. Cl. A61B 17/132

(52) U.S. Cl. CPC A61B 17/1325; A61B 17/1327

(57) ABSTRACT

An easy-to-use elastic limb tourniquet comprises a round elongated elastic body with a first end and a second end. The elongated elastic body has a diameter of at least 6 mm and a length between 50 cm and 120 cm. The first end of the elongated elastic body is equipped with a straight rigid guiding handle configured to facilitate threading of the tourniquet under the limb of a patient even when the limb is compressed by an external object. The rigid guiding handle allows self-application of the tourniquet with one arm. The second end forms an end loop with a sufficient internal opening to allow the guiding handle and the elongated elastic body to pass once therethrough. The second end further comprises a wedge clamp configured to retain the elongated elastic body when placed across thereof and while the tourniquet is under tension.

### CROSS REFERENCE DATA

This patent application is a continuation-in-part of my co-pending UA Patent Application No. U 2022 03433 filed on Sep. 19, 2022, and entitled “Hemostatic Tourniquet,” which is incorporated herein in its entirety by reference.

### BACKGROUND

Without limiting the scope of the invention, its background is described in connection with elastic tourniquets. More particularly, the invention describes an elastic tourniquet that is easy to apply with one hand.

Elastic tourniquets are a type of medical device used to stop bleeding in limbs, both in civilian and military applications. They are designed to be wrapped tightly around an injured limb, compressing the blood vessels and stopping blood flow to the area. This can be critical in situations where a person is bleeding heavily, as it can help prevent shock and even save their life.

In civilian settings, elastic tourniquets are often used in emergency medical situations, such as car accidents or other traumatic injuries. They are also commonly used by first responders, such as paramedics, as a way to quickly stop bleeding and stabilize patients before they can be transported to a hospital for more comprehensive medical care.

In military applications, elastic tourniquets are a vital tool for combat medics and other military personnel. In battlefield situations, injuries to limbs are common, and time is often of the essence when it comes to stopping bleeding and saving lives. Elastic tourniquets are an effective and portable way to quickly apply pressure to a wound and stop bleeding, allowing military personnel to provide critical care in even the most challenging environments.

One of the advantages of elastic tourniquets is their ease of use. Unlike traditional tourniquets, which can be difficult to apply correctly, elastic tourniquets can be easily applied by anyone, regardless of their level of medical training. They are also highly effective at stopping bleeding, with some models able to completely cut off blood flow to a limb in just seconds.

While elastic tourniquets can be highly effective in stopping bleeding in trauma and military applications, they are not without their limitations. Understanding these limitations is essential for ensuring that elastic tourniquets are used appropriately and safely in these settings.

One of the key limitations of elastic tourniquets is that they can cause damage to the tissues and nerves in the limb where they are applied. This is because the tourniquet restricts blood flow to the area, which can result in tissue ischemia and even tissue death. If the tourniquet is left on for too long, it can cause permanent damage to the limb and even lead to amputation. Since elastic tourniquets can be dangerous if left on for too long, the need exists for a tourniquet that can provide at least some indication of the duration of its use.

Another limitation of elastic tourniquets is that they can be difficult to apply correctly, especially in high-stress situations such as combat. If the tourniquet is not applied tightly enough, it may not be effective in stopping bleeding. On the other hand, if it is applied too tightly, it can cause damage to the tissues and nerves in the limb. Proper training and education are essential for ensuring that elastic tourniquets are applied correctly in these situations.

Overall, elastic tourniquets are an important medical tool for both civilian and military applications. They are highly effective at stopping bleeding in limbs and can be quickly and easily applied by anyone. However, the need exists for a tourniquet that can be easily applied, requires minimal training, and provides reliable compression to stop the bleeding in an emergency situation.

### SUMMARY

Accordingly, it is an object of the present invention to overcome these and other drawbacks of the prior art by providing a novel elastic limb tourniquet that is intuitive and easy to learn and use.

It is another object of the present invention to provide an elastic tourniquet that can be applied by one person, including a person experiencing bleeding—including application by one hand.

It is yet another objective of the present invention to allow rapid application of the elastic tourniquet to stop the bleeding as fast as possible.

It is a further object of the present invention to provide an elastic tourniquet that can be applied with minimal risk of unraveling and allowing for further bleeding.

It is yet another object of the invention to provide an elastic tourniquet that is compact and easy to fold.

The elastic tourniquet of the invention may include an elongated elastic body with a first end and a second end. The first end of the elongated elastic body may be equipped with a straight rigid guiding handle, which may have the same or similar cross-sectional shape and dimensions as that of the elongated elastic body. The guiding handle may be at least 7 cm long to facilitate threading the elongated elastic body under the compressed limb if needed. It may also be conveniently used to thread the elongated elastic body through the opening of the end loop on the second end of the tourniquet—such as during the application thereof around the bleeding limb. The straight rigid guiding handle is instrumental in the self-application of the tourniquet by an injured person when there is no outside help available.

The second end of the tourniquet is formed into an end loop protruding from a wedge clamp, which in turn is configured to retain the elongated elastic body when placed across thereof while the elongated elastic body is under tension.

The use of the tourniquet goes beyond stopping bleeding. The product may be used to affix the injured limb to a straight rigid support board or to apply external pressure to the limb during intravenous catheter insertion for delivery of IV medication.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through the use of the accompanying drawings, in which:

FIG. 1 is a perspective view of the elastic limb tourniquet of the present invention,

FIG. 2 is a side view of the same,

FIG. 3 is a top view of the same,

FIG. 4 is a perspective close-up view of the second end of the elongated elastic body,

FIG. 5 is a cross-sectional view of the same,

FIG. 6 is a top view of the straight rigid handle with indicia indicating the compression time, and

FIG. 7 is a top view of the guiding handle with a series of lights indicating the compression time.

### DETAILED DESCRIPTION OF PREFERRED EMBODIMENTS OF THE INVENTION

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

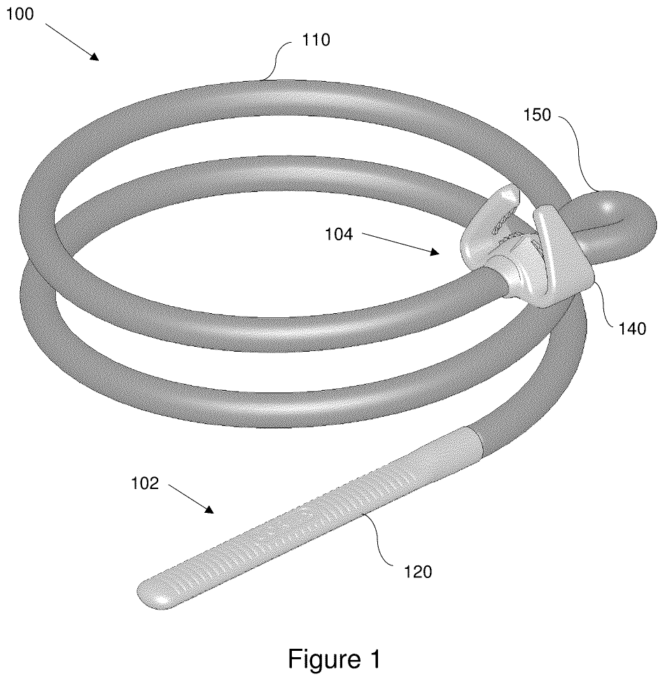

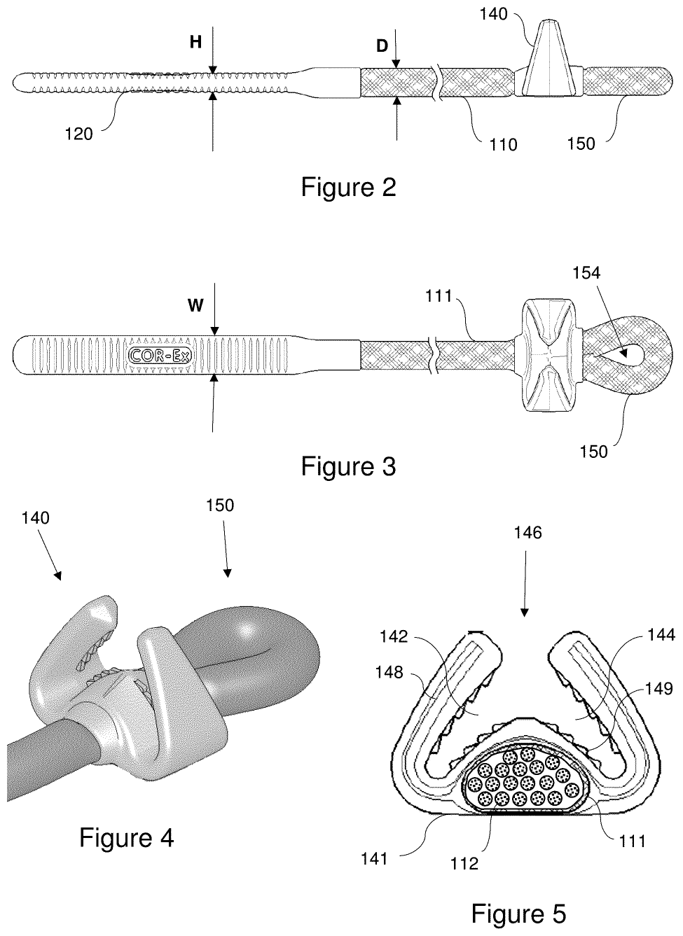

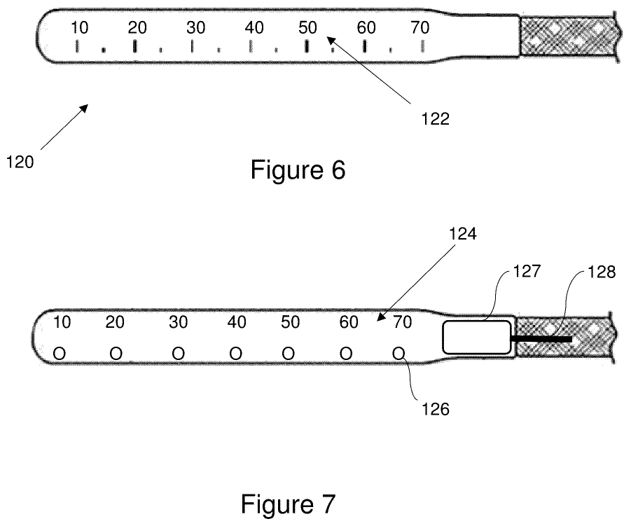

FIG. 1 shows a general perspective view of the elastic tourniquet 100 of the present invention. FIGS. 2 through 7 show further details of its design. The tourniquet 100 includes an elongated elastic body 110 with a first end 102 and a second end 104. The elongated elastic body 110 may be made from a variety of elastic materials such as latex or another natural or synthetic rubber, nylon, silicone, lycra, etc. The length of the elongated elastic body may be selected to be anywhere from about 50 cm to about 120 cm, such as at least 50 cm, at least 60 cm, at least 70 cm, at least 80 cm, at least 90 cm, at least 100 cm, at least 110 cm, or up to 120 cm. This length is generally sufficient to stretch and wrap the tourniquet around a limb such as a thigh at least two times to assure reliable hemostasis.

When pulled from one end, the length of the tourniquet increases as the pull tension is increased. In embodiments, the elasticity of the tourniquet may be selected to allow for a 2× length increase when pulled with a force of about 7 to 12 lbs.

The cross-section of the elastic elongated body may be round or close to round. In particular, the diameter of the elongated elastic body, when not stretched, may be at least 6 mm, at least 7 mm, at least 8 mm, at least 9 mm, at least 10 mm, at least 11 mm, or up to 15 mm. one advantage of making the elongated elastic body round is the ease of use and no risk of twisting in case of a non-round shape. Another advantage is the ease of positioning into the wedge clamp, as described below in greater detail, as the round shape does not require any particular orientation of the elongated elastic body to be concerned about.

In embodiments, the round elongated elastic body may have a smooth outside surface. In other embodiments, external features such as bumps, ridges, protrusions, or other patterns may be provided on the outside surface of the elongated elastic body aimed at increasing the friction resistance and improving the retention of the elongated elastic body once positioned in the wedge clamp.

In further embodiments, a stretchable fabric or polymer outer sleeve 111 (see FIG. 3) may be provided to surround the outside surface of the elongated elastic body to further increase the friction associated with the outside surface thereof. The fabric sleeve may be made with a knitted thread pattern to allow stretching and not limit the elongation of the elastic body 110 during use.

In further yet embodiments, the elongated elastic body may comprise a bundle of individual elastic strands 112—see FIG. 5. The strands may be intertwined with each other or collected and held together by virtue of the outer sleeve 111. The bundle of the elongated elastic sleeve may contain anywhere from 5 to 40 individual elastic strands, such as at least 5 elastic strands, at least 8 elastic strands, at least 10 elastic strands, at least 15 elastic strands, at least 20 elastic strands, at least 25 elastic strands, at least 30 elastic strands, at least 35 elastic strands, at least 40 elastic strands, or another number of elastic strands as the invention is not limited in this regard. While in some embodiments, all individual elastic strands may be the same, in other embodiments, individual elastic strands with larger diameters may be combined with elastic strands of smaller diameters—to make up the bundle of the elongated elastic body of at least 6 mm in diameter.

The first end 102 of the elongated elastic body 110 may be terminated in a straight ridged guiding handle 120—see FIGS. 1, 2, 3, 6, and 7. The guiding handle 120 is provided to facilitate administration of the tourniquet with one hand, and in particular, facilitate threading the elongated elastic body under a limb in case it is compressed by an external object and cannot be easily lifted. To achieve this objective, the rigid guiding handle may be made to have a cross-sectional shape to be similar to that of the elongated elastic body 110. In embodiments, the straight rigid guiding handle may have a cross-sectional area along at least an end portion thereof not exceeding the cross-sectional area of the elongated elastic body by more than 50 percent, or not exceeding it at all. The cross-sectional shape of the end portion of the guiding handle 120 may be that of an oval. The term “oval” includes any “flattened” circular shape such as a geometrical oval, an oblong shape, a shape of a rectangle with rounded corners, or another similar atraumatic smooth and flattened shape.

In embodiments, the straight rigid guiding handle 120 may have an oval cross-sectional shape along at least an end portion thereof having a width W exceeding the diameter D of the elongated elastic body and a height H being less than the diameter D of the elongated elastic body—see FIGS. 2 and 3. This makes the cross-sectional area of the straight rigid guiding handle 120 to be about the same as that of the elongated elastic body 110.

The length of the rigid guiding handle 120 may be sufficient to facilitate threading the tourniquet body 110 under the limb or to thread it through the opening 154 in the end loop 150—see FIG. 3. At the same time, there is no need to make it excessively long as it can interfere with the treatment of the patient and may catch on other items or clothing of the patient. Therefore, the length of the rigid guiding handle may be selected to be from about 7 cm to about 25 cm, such as at least 7 cm, at least 10 cm, at least 12 cm, at least 15 cm, at least 17 cm, at least 20 cm, at least 22 cm, or up to 25 cm.

It is important to record the occlusion time as it may alter the course of treatment for some patients. The rigid guiding handle 120 may have an elongated flattened area on one or both sides thereof containing indicia 122 suitable for recording the time when the tourniquet was applied or the duration of compression time. Suitable markings, ridges, or notches may be provided on the rigid handle 120 to indicate compression time. In other embodiments, a mechanical slider may be provided (not shown) to indicate compression time.

In further embodiments, the rigid guiding handle 120 may be equipped with an electronic timer 127 which may be embedded into the handle or otherwise attached thereto without increasing the cross-sectional area thereof—see FIG. 7. Such electronic timer 127 may be operatively connected to a series of lights, such as LED lights 126 spaced along the rigid handle 120. Upon activation, the timer 127 may be configured to cause each successive light 126 to illuminate as the compression time increases. In further embodiments, the timer may be automatically activated when the tourniquet 100 is stretched to be applied onto the limb. To achieve this, a pull thread 128 may extend from the timer 127 into the elastic elongated body 110, as seen in FIG. 7. Pulling on the handle may cause the extension of the pull thread 128, which may be transformed into an activation action of the timer 127. In some embodiments, the timer 127 may be configured to be activated once, while in other embodiments, activation and deactivation of the timer 127 and reset of the compression time indication may be caused by successive applications of a pull force above a predefined threshold.

The second end 104 of the elongated elastic body 110 may be formed into an end loop 150 with a sufficient internal opening 154 to allow the rigid guiding handle 120 followed by the elongated elastic body 110 to pass once therethrough. The second end 104 further comprises a wedge clamp 140 configured to retain the elongated elastic body 110 when placed across thereof and while the tourniquet 100 is under tension.

FIGS. 3 through 5 show the details of the second end of the tourniquet 100. The end loop 150 may be formed with the free end of the elongated elastic body embedded in or retained by the wedge clamp 140 located adjacent to the end loop 150. The wedge clamp 140 may, in turn, include a first V-shaped opening 142 with an open side 146 sized to be sufficiently large to accept the elongated elastic body 110 into the first V-shaped opening 142 from the open side thereof. The first V-shaped opening may have a shape tapering from an open side 146 to a size lower than the diameter of the elongated elastic body 110 so as to facilitate clamping on and retaining the elongated elastic body 110 within the wedge clamp 140 during use.

One significant advantage of the V-shaped opening design is that it allows for simple and rapid fixation of the tourniquet 100 on the limb with a single side movement to engage the taught elastic body 110 with the wedge clamp. This simplicity and ease of use are critically important to provide rapid hemostasis and avoid complex “tourniquet tightening” maneuvers required when other prior art tourniquets are deployed.

More than one V-shaped opening may be provided in the wedge clamp 140. An example of two V-shaped openings located side by side is seen in FIGS. 4 and 5. This may be useful to retain two portions of the elongated elastic body 110 when the tourniquet 100 is wrapped around the limb more than once. The first and the second V-shaped opening 142 and 144 may be located on the same side of the wedge clamp 140 so as to ensure a flat side 141 of the wedge clamp 140 can be located to face the limb of the patient.

The wedge clamp 140 may be made entirely from plastic or may have a metal insert 148, which may provide additional spring-like behavior when the elastic body 110 is wedged into the V-shaped opening.

In further embodiments, at least a portion or the entire internal area of the first or the second V-shaped openings 142 and 144 may include one or a series of protrusions 149, which may be shaped with a taper directing the elongated elastic body 110 into the wedge clamp 140 during use and preventing accidental release thereof prior to intended release of the tourniquet 100.

In use, the second end 104 may be placed in the visible vicinity of the limb to be occluded. The straight rigid guiding handle 102 may be used to wrap the tourniquet at least once (or multiple times) about the limb and thread the first end of the elongated elastic body through the opening 154 in the end loop 150. The tourniquet 100 may be pulled on and tightened about the limb, and optionally wrapped one or several times more about the limb.

The free portion of the elongated elastic body 110 may be wedged into the first V-shaped opening 142 of the wedge clamp 140 to be retained therein. The tourniquet 100 may be then wrapped about the limb again and pulled to apply compression to the limb. The free portion of the elongated elastic body 110 may be clamped again by wedging into the second V-shaped opening 144 to be retained until the time when the tourniquet 100 can be released. Pulling the tourniquet 100 during deployment causes its elongation of up to twice the original non-stretched length. Once deployed, the elastic tourniquet causes continuous compression on the limb, sufficient to stop arterial and venous bleeding downstream from the tourniquet. The simple and quick motion of “wrap and lock” allows for a rapid application and securing of the tourniquet to stop the bleeding from the limb.

It is contemplated that any embodiment discussed in this specification can be implemented with respect to any method of the invention, and vice versa. It will also be understood that particular embodiments described herein are shown by way of illustration and not as limitations of the invention. The principal features of this invention can be employed in various embodiments without departing from the scope of the invention. Those skilled in the art will recognize, or be able to ascertain, using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. Incorporation by reference is limited such that no subject matter is incorporated that is contrary to the explicit disclosure herein, no claims included in the documents are incorporated by reference herein, and any definitions provided in the documents are not incorporated by reference herein unless expressly included herein.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, Aft AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12, 15, 20 or 25%.

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. An elastic limb tourniquet comprising an elongated elastic body with a first end and a second end, the first end is equipped with a straight rigid guiding handle, the second end forms an end loop with a sufficient internal opening to allow the guiding handle followed by the elongated elastic body to pass once therethrough, the second end further comprising a wedge clamp configured to retain the elongated elastic body when placed across thereof and while under tension.

2. The elastic tourniquet, as in claim 1, wherein the length of the elastic body before stretching is selected to be sufficient to wrap about a limb of a subject at least two times.

3. The elastic tourniquet, as in claim 2, wherein the length of the elongated elastic body before stretching is between 50 cm and 120 cm.

4. The elastic tourniquet, as in claim 1, wherein the elongated elastic body is round in cross-section.

5. The elastic tourniquet, as in claim 4, wherein the elongated elastic body is at least 6 mm in diameter before stretching.

6. The elastic tourniquet, as in claim 1, wherein the elongated elastic body is contained within an outer sleeve configured to stretch along with the elongated elastic body during use and without limiting the elongation thereof.

7. The elastic tourniquet, as in claim 6, wherein the elongated elastic body comprises a bundle of individual elastic strands contained in the outer sleeve.

8. The elastic tourniquet, as in claim 1, wherein the straight rigid guiding handle has a cross-sectional area along at least an end portion thereof not exceeding the cross-sectional area of the elongated elastic body by more than 50 percent.

9. The elastic tourniquet, as in claim 8, wherein the straight rigid guiding handle has a cross-sectional area along at least an end portion thereof not exceeding the cross-sectional area of the elongated elastic body.

10. The elastic tourniquet, as in claim 1, wherein the straight rigid guiding handle has an oval cross-sectional shape along at least an end portion thereof having a width exceeding the diameter of the elongated elastic body and a height being less than the diameter of the elongated elastic body.

11. The elastic tourniquet, as in claim 1, wherein the straight rigid guiding handle has a length of at least 7 cm and not exceeding 25 cm to facilitate threading the elongated elastic body under a limb or through the end loop during applying the tourniquet around the limb.

12. The elastic tourniquet, as in claim 1, wherein the straight rigid guiding handle comprises indicia facilitating indication of limb compression time by the elastic tourniquet applied around the limb.

13. The elastic tourniquet, as in claim 12, wherein the indicia on the straight rigid guiding handle is a series of ridges or notches configured for marking the compression time thereon.

14. The elastic tourniquet, as in claim 1, wherein the indicia on the straight rigid guiding handle is a series of lights configured for indicating the compression time, wherein the straight rigid guiding handle further comprises a battery-operated timer built-in or associated therewith and configured once activated to turn on successive lights of the series of lights according to a time since activation of the timer.

15. The elastic tourniquet, as in claim 14, wherein the timer is further configured to self-activate upon stretching of the elongated elastic body above a predefined elongation threshold.

16. The elastic tourniquet, as in claim 1, wherein the wedge clamp comprises a first V-shaped opening with an open side sized to be sufficiently large to accept the elongated elastic body into the first V-shaped opening from the open side thereof, the first V-shaped opening tapering to a size lower than the diameter of the elongated elastic body to facilitate clamping on and retaining the elongated elastic body within the wedge clamp during use.

17. The elastic tourniquet as in claim 16, wherein the V-shaped opening is configured to support rapid application of the elastic tourniquet with a “wrap and lock” motion.

18. The elastic tourniquet, as in claim 1, wherein the wedge clamp further comprises a second V-shaped opening positioned next to and sized the same as the first V-shaped opening, both V-shaped openings are positioned on the same side of the wedge clamp and configured to correspondingly retain a first portion the elongated elastic body and the second portion thereof within the wedge clamp, thereby the elastic limb tourniquet may be wrapped at least twice and retained about the limb.

19. The elastic tourniquet, as in claim 18, wherein at least a portion of internal area of the first or the second V-shaped opening comprises a series of protrusions directing the elongated elastic body into the wedge clamp.
